(19) United States
(12) Patent Application Publication
(10) Pub. No.: US 2025/0262452 A1
(43) Pub. Date: Aug. 21, 2025

(54) LIGHT DIFFUSION DEVICE

(71) Applicant: FURUKAWA ELECTRIC CO., LTD., Tokyo (JP)

(72) Inventors: Hideaki HASEGAWA, Tokyo (JP); Junichi HASEGAWA, Tokyo (JP)

(21) Appl. No.: 19/199,388

(22) Filed: May 6, 2025

Related U.S. Application Data
(63) Continuation of application No. PCT/JP2023/039804, filed on Nov. 6, 2023.

(30) Foreign Application Priority Data
Nov. 11, 2022 (JP) 2022-180691
Nov. 11, 2022 (JP) 2022-180692

(51) Int. Cl. A61N 5/06

(52) U.S. Cl. CPC A61N 5/062; A61N 2005/063; A61N 2005/0665

(57) ABSTRACT

A light diffusion device comprises: an optical transmission cable which transmits a laser beam emitted from a laser oscillator and outputs the transmitted laser beam from an output surface at a leading end thereof; and a covering layer which has a function of absorbing the laser beam and/or a function of diffusing light and which covers the optical transmission cable. The covering layer has a leading end that protrudes in the output direction of light by a length required for cutting off the peripheral portion of said light.

This application is based on and claims the benefit of priority to Japanese Patent Application No. 2022-180691 and 2022-180692 filed on Nov. 11, 2022 and is a Continuation application of PCT Application No. PCT/JP2023/039804 filed on Nov. 6, 2023. The entire contents of each application are hereby incorporated herein by reference.

### TECHNICAL FIELD

The present invention relates to a light diffusion device.

### BACKGROUND ART

Conventionally, in the medical field, a light diffusion device has been used for insertion into a human body to irradiate cells with light. For example, Patent Document 1 discloses a device including a fiber core, a cladding enclosing the fiber core, an open cavity, and a covering.

### CITATION LIST

### Patent Document

Patent Document 1: Japanese Unexamined Patent Application, Publication No. 2020-72969

### DISCLOSURE OF THE INVENTION

### Problems to be Solved by the Invention

Incidentally, a light diffusion device is used, for example, in photoimmunotherapy and photodynamic therapy, which are cancer treatment methods,

for inserting the distal end side of an optical transmission cable into a human body to irradiate laser light onto a drug that has been administered to the human body and reached cancer cells. At this time, from the viewpoint of treatment efficiency, it is desirable to make the light intensity distribution of the laser light emitted from the optical transmission cable more uniform in a region within a predetermined radius from the center of the laser light. In the device of Patent Document 1, although light having a flat-top light intensity distribution can be irradiated, it is necessary to provide a sealed open cavity or the like between the covering and the cladding, and there is room for improvement in terms of processability.

An object of the present invention is to provide a light diffusion device that can be easily produced and can irradiate light with a flat-top light intensity distribution.

### Means for Solving the Problems

(1) A light diffusion device includes an optical transmission cable configured to transmit light emitted from a light source and emit the transmitted light from an emission surface of a distal end part, and a covering layer that has at least one of a function of absorbing the light or a function of scattering the light and that covers the optical transmission cable. The covering layer has a distal end part that protrudes by a required length for cutting a peripheral edge part of the light, in a direction in which the light is emitted.

(2) In the light diffusion device according to (1), the optical transmission cable includes a core and a cladding formed on an outer periphery of the core. The required length is calculated by the following Equation (1):

$$Y = {( 1 / {NA}^{2} - 1 )}^{1 / 2} \times d 2 \qquad Equation ( 1 )$$

where Y is the required length, NA is an aperture coefficient of the optical transmission cable, and d2 is a thickness of the cladding.

(3) In the light diffusion device according to (1) or (2), the optical transmission cable includes a core and a cladding formed on an outer periphery of the core. The cladding has a thickness of 1/10 or less of an outer diameter of the core. The covering layer has a thickness greater than that of the cladding.

(4) In the light diffusion device according to any one of (1) to (3), the emission surface of the optical transmission cable is inclined with respect to an axial direction of the optical transmission cable.

(5) In the light diffusion device according to any one of (1) to (4), the covering layer has a refractive index equal to or greater than a refractive index of a covering material of the optical transmission cable.

(6) In the light diffusion device according to any one of (1) to (5), wherein the covering layer has a refractive index of 1.53 or more.

(7) The light diffusion device according to any one of (1) to (6) further includes a reflective member having a refractive surface that refracts the light emitted from the emission surface, and a tubular member made of resin into which the optical transmission cable and the reflective member are inserted. The refractive surface is disposed at a predetermined distance from the emission surface in the tubular member and is inclined with respect to an axial direction of the optical transmission cable. The light emitted from the emission surface is emitted in a direction that is inclined at a predetermined angle or more with respect to the axial direction of the optical transmission cable.

(8) In the light diffusion device according to any one of (1) to (7), the reflective member is a rod-shaped member made of quartz or silicon and is spaced apart from the optical transmission cable in the tubular member. The refractive surface is formed on an end part of the rod-shaped member on a side adjacent to the optical transmission cable.

(9) In the light diffusion device according to any one of (1) to (8), the refractive surface includes a vapor-deposited metal.

(10) In the light diffusion device according to any one of (1) to (9), the optical transmission cable is a plastic fiber including a core having an outer diameter of 500 μm or more, and a cladding made of resin and formed on an outer periphery of the core. An outer diameter of the refractive surface viewed from the axial direction of the optical transmission cable is larger than the outer diameter of the core.

(11) In the light diffusion device according to any one of (1) to (10), wherein an unevenness of the refractive surface on which the light is incident is equal to or less than a wavelength of the light generated from the light source.

(12) In the light diffusion device according to any one of (1) to (11), the refractive surface is formed in a curved surface shape that is concave with respect to the emission surface.

### Effects of the Invention

According to the present invention, it is possible to provide a light diffusion device that can be easily produced and can irradiate light with a flat-top light intensity distribution.

### BRIEF DESCRIPTION OF THE DRAWINGS

FIG. 1 is a side view schematically showing an appearance of a light diffusion device according to a first embodiment;

FIG. 2 is a longitudinal sectional view schematically showing the light diffusion device according to the first embodiment;

FIG. 3 is a cross-sectional view taken along line III-III of FIG. 1;

FIG. 4 is a longitudinal sectional view schematically showing a light diffusion device according to a second embodiment;

FIG. 5 is a side view schematically showing an appearance of a light diffusion device according to a third embodiment;

FIG. 6 is a longitudinal sectional view schematically showing the light diffusion device according to the third embodiment;

FIG. 7 schematically shows a light diffusion device according to a fourth embodiment, and is a side view of the light diffusion device mainly irradiating laser light to the side;

FIG. 8 schematically shows a light diffusion device according to the fourth embodiment, and is a side view of the light diffusion device that mainly irradiates laser light rearward;

FIG. 9 is a side view schematically showing a light diffusion device according to a fifth embodiment;

FIG. 10 is a side view schematically showing a light diffusion device according to a sixth embodiment;

FIG. 11 is a side view schematically showing a light diffusion device according to a seventh embodiment;

FIG. 12 is a side view schematically showing a light diffusion device according to an eighth embodiment;

FIG. 13 is a graph showing a light intensity distribution of Example 1;

FIG. 14 is a graph showing a light intensity distribution of Example 2; and

FIG. 15 is a graph showing a light intensity distribution of a comparative example.

### PREFERRED MODE FOR CARRYING OUT THE INVENTION

Hereinafter, embodiments of the present invention will be described with reference to the drawings. The present invention is not limited to the following embodiments. The drawings referred to in the following description merely schematically show shapes, sizes, and positional relationships so that the contents of the present disclosure can be understood. That is, the present invention is not limited to only the shapes, sizes, and positional relationships illustrated in the drawings.

### First Embodiment

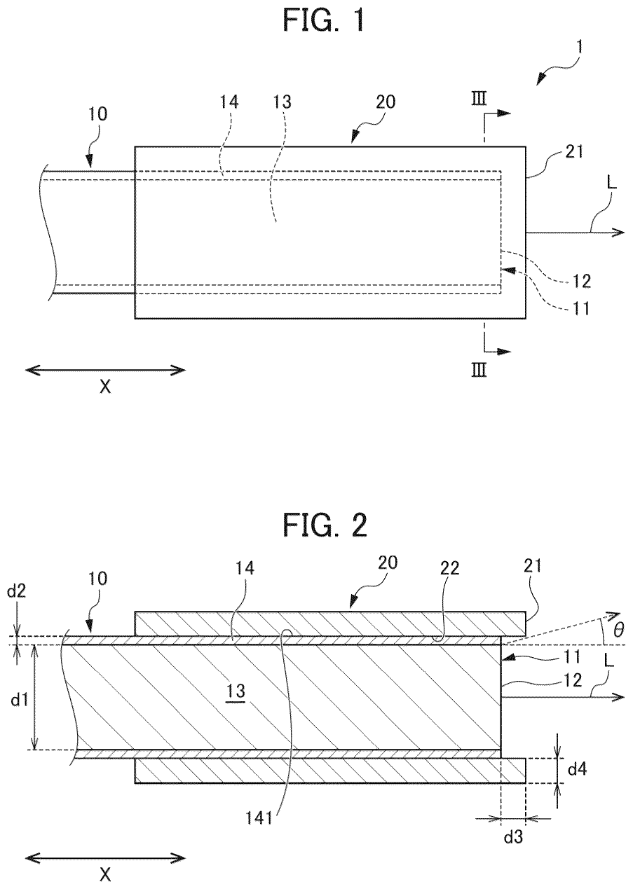

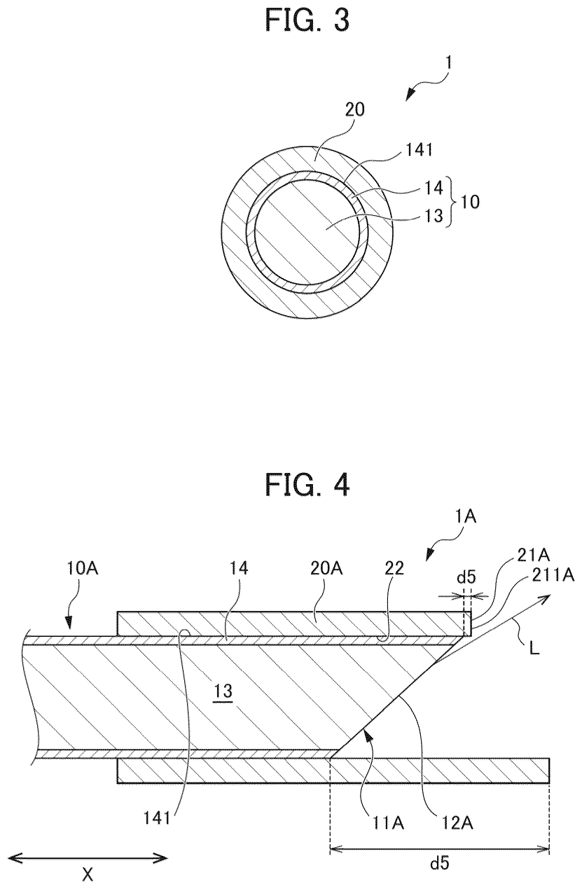

A light diffusion device 1 according to a first embodiment of the present invention will be described with reference to FIGS. 1 to 3. FIG. 1 is a side view of the light diffusion device 1. FIG. 2 is a longitudinal sectional view of the light diffusion device 1. FIG. 3 is a cross-sectional view taken along line III-III shown in FIG. 1. In FIG. 1, an optical transmission cable 10 covered with a covering layer 20 and a core 13 in the optical transmission cable 10 are indicated by broken lines.

The light diffusion device 1 of the present embodiment is mounted on a medical device for performing photoimmunotherapy, which is a method for treating cancer. The photoimmunotherapy treats cancer by administering to a human body a drug composed of an antibody that binds to cancer cells and a substance that reacts to light, and then irradiating the drug bound to the cancer cells with laser light L to destroy the cancer cells. The light diffusion device 1 is inserted into, for example, a conduit provided in an endoscope, and is used in a state where the distal end part thereof is exposed to the outside. The present invention is not limited to photoimmunotherapy, and can be used for photodynamic therapy.

As shown in FIGS. 1 and 2, the light diffusion device 1 includes a laser oscillator (not shown) as a light source, an optical transmission cable 10, and a covering layer 20. The covering layer may be a resin or a metal in which a part of light is scattered by slight unevenness on the order of wavelength on the surface.

The laser oscillator includes a semiconductor laser, generates laser oscillation by energizing the semiconductor laser, and generates laser light L. The laser oscillator generates red laser light L having a wavelength of 600 nm or more and 700 nm or less.

The optical transmission cable 10 is an optical fiber cable having an optical transmission path through which the laser light L emitted from the laser oscillator is transmitted. The laser oscillator is disposed on the proximal end part side of the optical transmission cable 10, and the covering layer 20 is provided on a distal end part 11 side. The optical transmission cable 10 transmits the laser light L generated in the laser oscillator through the optical transmission path and emits the laser light L from an emission surface 12 at the distal end part 11. The emission surface 12 of the present embodiment is a surface perpendicular to the axial direction X of the optical transmission cable 10. The axial direction X of the optical transmission cable 10 in the present specification means the axial direction of the optical transmission cable 10 at the distal end part 11.

The optical transmission cable 10 according to the present embodiment is a plastic fiber, and includes the core 13 and a cladding 14 made of resin, formed on the outer periphery of the core 13. Examples of the resin forming the cladding 14 include PTFE and PVDF. In the present embodiment, the emission surface 12 of the optical transmission cable 10 is the surface of the core 13 at the distal end part 11. The laser light L is emitted so that its optical axis is parallel to the axial direction X of the optical transmission cable 10.

The outer diameter d1 of the core 13 of the optical transmission cable 10 is preferably 250 μm or more. In the present embodiment, the outer diameter d1 of the core 13 is 500 μm. The optical transmission cable 10 of the present embodiment is a single-core optical fiber, but may be a multi-core optical fiber. The shape of the core 13 may be an ellipse or a rectangle other than a perfect circle when viewed from the axial direction X of the optical transmission cable 10. The optical transmission cable 10 may be an optical fiber made of quartz in which the core 13 and the cladding 14 are made of quartz, or may be a polymer clad optical fiber in which the core 13 is made of quartz and the cladding 14 is made of resin. Examples of the quartz-based material forming the core include quartz in which the core is not doped with impurities and quartz doped with germanium. Examples of the resin forming the cladding include fluorine-based resins such as PTFE, PVDF, and ETFE, polyimide, silicone, and copolymers thereof.

The thickness d2 of the cladding 14 is preferably 1/10 or less of the outer diameter d1 of the core 13 from the viewpoint of reducing the diameter of the optical transmission cable 10 while efficiently emitting the laser light L. In the present embodiment, the thickness d2 of the cladding 14 is 25 μm. Although the laser light L is transmitted through the core 13, a part of the laser light L reflected by the cladding 14 may leak to the cladding 14 and propagate as cladding mode light. The optical transmission cable 10 includes a covering material (not shown) that covers the cladding 14 to protect the optical transmission cable 10 itself.

The covering layer 20 has at least one of a function of absorbing the laser light L or a function of scattering the laser light L. For example, the covering layer 20 has a refractive index higher than that of the cladding 14, and absorbs or scatters the cladding mode light leaking from the cladding 14 or the laser light L emitted from the emission surface 12.

The covering layer 20 covers at least the distal end part 11 side of the optical transmission cable 10. The covering layer 20 of the present embodiment is formed of a cylindrical tube made of resin. The covering layer 20 covers the cladding 14 in a state where the inner peripheral surface 22 thereof is in contact with the covering material that covers the outer peripheral surface 141 of the cladding 14. The covering layer 20 extends in a direction in which the laser light L is emitted beyond the distal end part 11 of the optical transmission cable 10. That is, a distal end part 21 of the covering layer 20 in the axial direction X of the optical transmission cable 10 is located so as to protrude past the distal end part 11 in the direction in which the laser light L is emitted. In the present embodiment, the covering layer 20 extends in a direction in which the laser light L is emitted beyond the distal end part 11 of the optical transmission cable 10. That is, the distal end part 21 of the covering layer 20 is located at a position beyond the distal end part 11 of the optical transmission cable 10 in the direction in which the light is emitted.

Here, from the viewpoint of the treatment efficiency, the light intensity distribution of the laser light L emitted from the emission surface 12 is desirably a flat top intensity distribution in which the variation of the light intensity in a range of a predetermined radius from the center of the laser light L is small, and the light intensity sharply decreases beyond the range of the predetermined radius, rather than an intensity distribution close to a Gaussian distribution. In the light diffusion device 1 of the present embodiment, the covering layer 20 covering the outer periphery of the optical transmission cable 10 absorbs or scatters the laser light L or the cladding mode light emitted from the distal end part 11 in a direction inclined obliquely with respect to the axial direction X of the optical transmission cable 10, thereby realizing a flat-top light intensity distribution.

The distal end part 21 of the covering layer 20 protrudes in the direction in which the laser light L is emitted by a required length Y for cutting off the peripheral edge part of the laser light L. The required length Y indicates the distance d3 between the distal end part 21 of the covering layer 20 and the distal end part 11 of the optical transmission cable 10 in the axial direction X of the optical transmission cable 10 in FIG. 2. The required length Y may be calculated by the following Equation (1) using the aperture coefficient NA of the optical transmission cable 10 and the thickness d2 of the cladding 14, for example. Y=(1/NA²−1)^{1/2}×d2 Equation (1)

The above Equation (1) is obtained from the following Equation (2). That is,

$$NA = sin θ = d 2 / ( d {2}^{2} + {Y}^{2} ) \qquad Equation ( 2 )$$

As shown in FIG. 2, θ is a spread angle of the laser light L emitted from the emission surface 12. That is, by protruding the covering layer 20 in the direction in which the laser light L is emitted by the length Y obtained by the above Equation (1), it is possible to cut the light that spreads at a wider angle with respect to the optical axis of the laser light L than the spread angle of the laser light L. Therefore, while maintaining the intensity of the laser light, the light intensity distribution within a predetermined radius from the center of the laser light L can be made more uniform, and flat-top light can be irradiated. The above Equation (1) can be applied to a single-core optical fiber or a multi-core optical fiber.

TABLE 1

| Type of | | | Aperture | Refractive index | Protruding amount |
|---|---|---|---|---|---|
| optical fiber | Core | Cladding | coefficient | of cladding | of covering layer |
| Plastic fiber | Methacrylic resin | Fluorine-based resin | 0.485~0.50 | 1.35 | 18 μm< |
| Polymer clad fiber | Quartz | Polymer | 0.37~0.43 | 1.35 | 25 μm< |
| Polymer clad fiber | Quartz | Silicone | 0.37~0.43 | 1.43 | 25 μm< |
| Optical fiber | Quartz | Quartz | MM0.2~0.275 | 1.375~1.72 | 49 μm< |
| made of quartz | | | | | |

Table 1 shows the relationship between the type of the optical fiber, i.e., the type of the optical transmission cable 10, the aperture coefficient NA, the types of the materials forming the core 13 and the cladding 14, etc., and the protruding amount of the covering layer 20, which is the length Y obtained by the above Equation (1). Table 1 shows the protruding amount of the covering layer 20 when the thickness d2 of the cladding 14 is 10 μm. As shown in Table 1, for example, in a plastic fiber in which the core 13 is formed of a methacrylic resin and the cladding 14 is formed of a fluorine-based resin, the protruding amount of the covering layer 20 obtained by the above Equation (1) is 18 μm. By using the above Equation (1), it is possible to obtain an appropriate protruding amount of the covering layer 20 for forming flat-top light according to the type of the optical fiber. It is also possible to cut the light on the peripheral edge part side of the laser light according to the application of the optical fiber based on the obtained protruding amount. In this case, although the entire intensity of the emitted light decreases, the light is more flat-topped.

The thickness d4 of the covering layer 20 is preferably larger than the thickness d2 of the cladding 14. The thickness d4 of the covering layer 20 is preferably 1/10 of the outer diameter d1 of the core 13 and about twice the thickness d2 of the cladding 14 from the viewpoint of concentrating the light intensity distribution toward the center side while suppressing the thickness of the light diffusion device 1 in the radial direction. In the present embodiment, the thickness d4 of the covering layer 20 is 50 μm.

The tube made of resin, forming the covering layer 20 may be, for example, a nylon tube, a polytetrafluoroethylene (PTFE) tube, or a tube having an inner layer formed of PTFE and an outer layer formed of polyimide (hereinafter referred to as a PTFE/polyimide tube). The PTFE/polyimide tube may have, for example, a thickness of 25 μm of a PTFE layer as an inner layer and a thickness of 25 μm of a polyimide layer as an outer layer. The term “nylon tube” includes both tubes composed of nylon only and tubes composed mainly of nylon, and the term “PTFE tube” includes both tubes composed of PTFE only and tubes composed mainly of PTFE. In the present embodiment, the covering layer 20 is formed of a nylon tube.

In addition to the above, examples of the resin forming the covering layer 20 include fluorine-based resins other than PTFE, such as ETFE, silicone resins, polymethyl methacrylate resins, acrylic resins, epoxy resins, and polycarbonates. The refractive index of the resin forming the covering layer 20 is 1.35 for ETFE, 1.43 for silicone resin, 1.49 for polymethyl methacrylate resin, 1.50 for acrylic resin, 1.53 for nylon resin, 1.57 for epoxy resin, and 1.59 for polycarbonate. The refractive index of the covering layer 20 is preferably equal to or greater than the refractive index of the covering material of the optical transmission cable 10. The refractive index of the covering layer 20 is preferably 1.53 or more. The refractive indexes are values obtained by a method in accordance with JIS K7142:2014.

### Second Embodiment

Next, a light diffusion device 1A according to a second embodiment will be described with reference to FIG. 4. FIG. 4 is a sectional view schematically showing the light diffusion device 1A according to the second embodiment. In the following description of the second embodiment, components corresponding to those of the first embodiment are denoted by corresponding reference numerals based on the same conventions. Their descriptions may be omitted or incorporated.

The light diffusion device 1A of the present embodiment includes a laser oscillator (not shown), an optical transmission cable 10A, and a covering layer 20A. The light diffusion device 1A according to the present embodiment differs from the light diffusion device 1 according to the first embodiment mainly in the features of the distal end part sides of the optical transmission cable and the covering layer.

The distal end part 11A of the optical transmission cable 10A is formed by cutting obliquely with respect to the axial direction X of the optical transmission cable 10A. That is, an emission surface 12A of the distal end part 11A is inclined with respect to the axial direction X of the optical transmission cable 10. Accordingly, as shown in FIG. 4, laser light L emitted from the emission surface 12A is emitted in a direction (in FIG. 4, an obliquely upper right direction in the figure) inclined at a predetermined angle or more with respect to the axial direction X of the optical transmission cable 10A.

The covering layer 20A is formed of a cylindrical tube made of resin. A distal end part 21A of the covering layer 20A is formed by cutting the cylindrical tube obliquely with respect to the axial direction X of the optical transmission cable 10A. That is, the distal end part 21A is inclined with respect to the axial direction X of the optical transmission cable 10A. Specifically, the distal end part 21A of the covering layer 20A is formed such that a part 211A located on the side in the direction in which the laser light L is emitted (hereinafter, referred to as an emission side part) is located closest to the proximal end part side of the optical transmission cable 10A and the distal end part 21A extends in a direction away from the optical transmission cable 10A in the axial direction X of the optical transmission cable 10A as the distance from the emission side part 211A increases. In other words, the distance d5 between the distal end part 21A and the distal end part 11A in the axial direction X of the optical transmission cable 10A increases as the distance from the emission side part 211A increases. That is, the distal end part 21A is inclined in a direction opposite to the distal end part 11A of the optical transmission cable 10A. The covering layer 20 is formed at a position where at least the centers of the laser light L emitted from the emission surface 12A do not overlap.

The emission side part 211A of the distal end part 21A of the covering layer 20A protrudes by a required length Y for cutting the peripheral edge part of the laser light L in the direction in which the laser light L is emitted. In FIG. 4, the required length Y indicates the distance d5 between the emission side part 211A of the distal end part 21A of the covering layer 20A and the emission side part 211A of the distal end part 11A of the optical transmission cable 10A in the axial direction X of the optical transmission cable 10A. The required length Y may be calculated by the above Equation (1).

### Third Embodiment

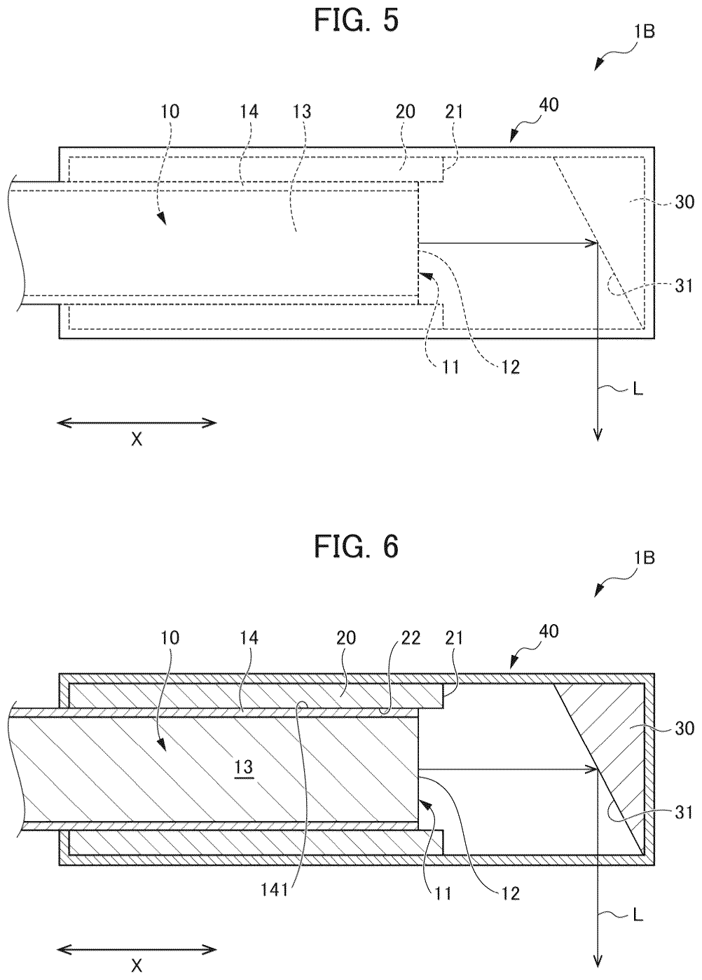

Next, a light diffusion device 1B according to a third embodiment will be described with reference to FIGS. 5 and 6. FIG. 5 is a side view schematically showing an appearance of the light diffusion device 1B according to the third embodiment. FIG. 6 is a sectional view schematically showing the light diffusion device 1B according to the third embodiment. In the following description of the third embodiment, components corresponding to those of the first embodiment are denoted by corresponding reference numerals based on the same conventions. Their descriptions may be omitted or incorporated.

The light diffusion device 1B of the present embodiment includes a laser oscillator (not shown), an optical transmission cable 10, a covering layer 20, a refractive member 30 as a reflective member, and a holding member 40. The light diffusion device 1B of the present embodiment mainly differs from that of the first embodiment in that it includes the refractive member 30 and the holding member 40 as a tubular member.

The refractive member 30 is a lens that refracts laser light L emitted from an emission surface 12 of the optical transmission cable 10. The refractive member 30 is disposed to be spaced apart from a distal end part 11 in the axial direction X of the optical transmission cable 10.

A refractive surface 31 is formed on the optical transmission cable 10 side of the refractive member 30. The refractive surface 31 faces the emission surface 12 and is disposed so as to be inclined with respect to the axial direction X of the optical transmission cable 10. As shown in FIG. 6, the refractive surface 31 emits the laser light L emitted from the emission surface 12 at the distal end part 11 of the optical transmission cable 10 to the outside of the holding member 40 in a direction that is inclined at an angle of a predetermined angle or more with respect to the axial direction X of the optical transmission cable 10.

The holding member 40 is a cylindrical tube. Both ends of the holding member 40 in the axial direction are sealed in a state in which the optical transmission cable 10, the covering layer 20, and the refractive member 30 are housed therein.

A window (not shown) through which the laser light L passes may be formed on the outer periphery of the holding member 40. The window of the holding member 40 is formed at a position where the laser light L is emitted on the outer periphery of the holding member 40. For example, the window may be an opening whose diameter is smaller than the outer diameter d1 of a core 13, or may be a plurality of small holes. As a result, the peripheral edge part of the laser light L can be cut, and the laser light L having a more flat-topped light intensity distribution can be irradiated.

The optical transmission cable 10, the covering layer 20, and the refractive member 30 are fixed in the holding member 40 by, for example, setting the outer diameters or the widths thereof to be larger than the inner diameter of the holding member 40 and tightening them with a force directed radially inward by the holding member 40 (in a so-called interference fit state). The material of the holding member 40 preferably has a light transmittance of 50% or more. Examples of the material of the holding member 40 include acrylic resin and FEP (fluororesin formed by combining tetrafluoroethylene and hexafluoropropylene).

### Fourth Embodiment

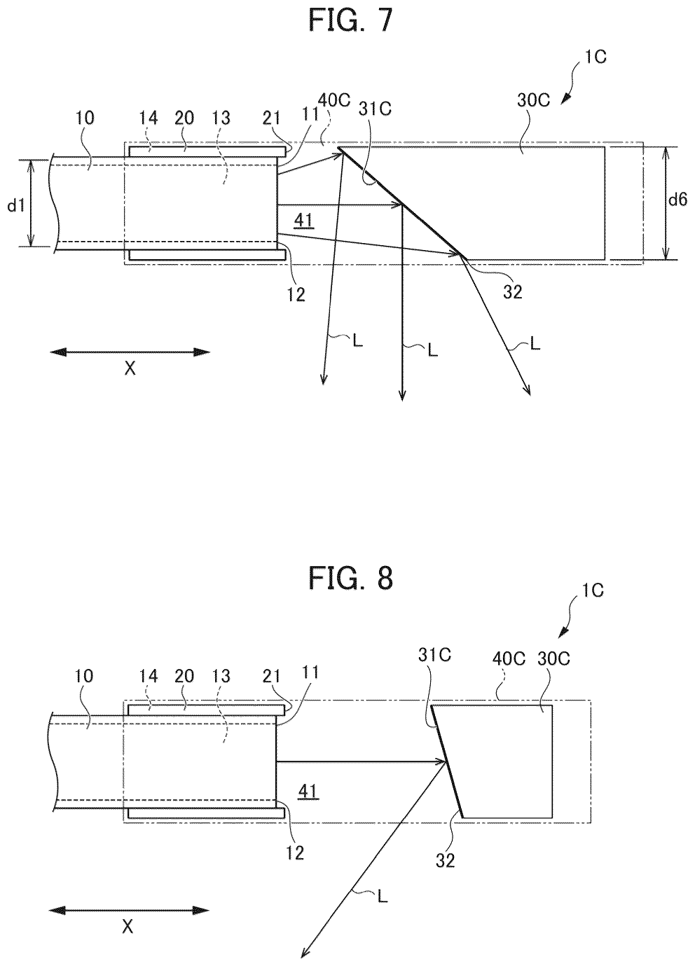

Next, a light diffusion device 1C according to a fourth embodiment will be described with reference to FIGS. 7 and 8. FIG. 7 schematically shows the light diffusion device 1C according to the fourth embodiment, and is a side view of the light diffusion device 1C that mainly irradiates the laser light L to the side. FIG. 8 schematically shows a light diffusion device 1C according to the fourth embodiment, and is a side view of the light diffusion device 1C that mainly irradiates the laser light L rearward. In FIGS. 7 and 8, a tubular member 40C is indicated by a two-dot chain line. In the following description of the fourth embodiment, components corresponding to those of the first embodiment are denoted by corresponding reference numerals based on the same conventions. Their descriptions may be omitted or incorporated.

The light diffusion device 1C of the present embodiment includes a laser oscillator (not shown), an optical transmission cable 10, a covering layer 20, a rod-shaped member 30C as a reflective member, and a tubular member 40C. The light diffusion device 1C of the present embodiment mainly differs from that of the first embodiment in that it includes the rod-shaped member 30C and the tubular member 40C.

The tubular member 40C is a cylindrical tube made of resin. The term “tube made of resin” herein includes both tubes made of resin only and tubes made mainly of resin. The tubular member 40C houses a part of the optical transmission cable 10, the covering layer 20, and the rod-shaped member 30C therein. The tubular member 40C is configured to be reducible in diameter. In the present embodiment, the optical transmission cable 10 is inserted into the tubular member 40C such that at least a distal end part 11 side is located inside the tubular member 40C. As shown in FIG. 7, the optical transmission cable 10 is housed in the tubular member 40C in a state of extending in the axial direction of the tubular member 40C. The resin forming the tubular member 40C preferably has a light transmittance of 50% or more. Examples of the resin forming the tubular member 40C include polyimide, FEP (tetrafluoroethylene-hexafluoropropylene copolymer), and acrylic resin.

The rod-shaped member 30C is made of quartz and is housed in the tubular member 40C. The term “rod-shaped member 30C made of quartz” herein includes both a rod-shaped member 30C made of quartz only and a rod-shaped member 30C mainly made of quartz. Specifically, the rod-shaped member 30C is housed in the tubular member 40C, extending in the axial direction of the tubular member 40C and spaced apart from the optical transmission cable 10. In the present embodiment, the rod-shaped member 30C is disposed substantially coaxially with the optical transmission cable 10 in the tubular member 40C. The rod-shaped member 30C is entirely housed in the tubular member 40C and is not exposed to the outside. The optical transmission cable 10 and the rod-shaped member 30C are fixed in the tubular member 40C by, for example, setting the outer diameters thereof to be larger than the inner diameter of the tubular member 40C and tightening them with a force directed radially inward by the tubular member 40C (in a so-called interference fit state). The rod-shaped member 30C may be made of silicon. The term “rod-shaped member 30C made of silicon” herein includes both a rod-shaped member 30C made of silicon only and a rod-shaped member 30C mainly made of silicon.

A refractive surface 31C is formed at an end part of the rod-shaped member 30C on the optical transmission cable 10 side. The refractive surface 31C is an inclined surface made of quartz, formed by cutting the rod-shaped member 30C obliquely with respect to the axial direction thereof. The term “refractive surface 31C made of quartz” herein includes both a refractive surface 31C made of quartz only and a refractive surface 31C mainly made of quartz. The refractive surface 31C faces an emission surface 12 in the tubular member 40C and is disposed so as to be inclined with respect to the axial direction X of the optical transmission cable 10. The refractive surface 31C may be made of silicon. The term “refractive surface 31C made of silicon” herein includes both a refractive surface 31C made of silicon only and a refractive surface 31C mainly made of silicon.

As shown in FIG. 7, the refractive surface 31C emits the laser light L emitted from the emission surface 12 at the distal end part 11 of the optical transmission cable 10 to the outside of the tubular member 40C in a direction that is inclined at a predetermined angle or more with respect to the axial direction X of the optical transmission cable 10. At this time, the laser light L having a flat-top light intensity distribution is irradiated by the distal end part 21 of the covering layer 20 protruding so as to cut the peripheral edge part of the laser light L in the direction in which the laser light L is emitted. For example, as shown in FIG. 7, the refractive surface 31C refracts each laser light L emitted in the axial direction X of the optical transmission cable 10 from a plurality of locations of the emission surface 12, and emits the laser light L to the side of the tubular member 40C. For example, the laser light L having a flat-top light intensity distribution refracted through the refractive surface 31C passes through the tubular member 40C, is emitted in a direction inclined with respect to the insertion direction of the optical transmission cable 10, and is irradiated onto cancer cells or the like present on the surface of an organ. Alternatively, for example, as shown in FIG. 8, the inclination of the refractive surface 31C may be set to be closer to the vertical direction with respect to the axial direction X of the optical transmission cable 10 than the refractive surface 31C shown in FIG. 7. With this configuration, as shown in FIG. 8, the laser light L having a flat-top light intensity distribution can be irradiated rearward from the refractive surface 31C.

As shown in FIG. 7, the refractive surface 31C of the present embodiment is formed in a planar shape as a whole. The unevenness of the refractive surface 31C on which the laser light L is incident is preferably equal to or less than the wavelength of the laser light L generated from the laser oscillator. For example, by mirror-polishing the refractive surface 31C, it is possible to realize an unevenness equal to or less than the wavelength of the laser light L. A metal 32 is vapor-deposited on the refractive surface 31C of the present embodiment. Examples of the metal 32 vapor-deposited on the refractive surface 31C include gold, silver, and aluminum.

As shown in FIG. 7, the outer diameter d6 of the rod-shaped member 30C is larger than the outer diameter d1 of the core of the optical transmission cable 10. That is, the outer diameter of the refractive surface 31 viewed from the axial direction X of the optical transmission cable 10 is larger than the outer diameter d1 of the core. With this configuration, since the refractive surface 31C that receives the laser light L emitted from the optical transmission cable 10 is larger than the emission surface 12, it is possible to allow for misalignment of the position of the refractive surface 31C with respect to the optical transmission cable 10.

The refractive surface 31C is disposed at a predetermined distance from the emission surface 12 in the tubular member 40C. The distance between the emission surface 12 and the refractive surface 31C is preferably in the range of 0.5 mm to 1 mm. A medium having a refractive index different from those of the emission surface 12 and the refractive surface 31C exists between the emission surface 12 and the refractive surface 31C. For example, in the present embodiment, only space 41 exists as the medium having a different refractive index between the emission surface 12 and the refractive surface 31C. A lens or the like having a refractive index different from those of the emission surface 12 and the refractive surface 31C and being in contact with both the emission surface 12 and the refractive surface 31C may be interposed between the emission surface 12 and the refractive surface 31C so as to fill the space 41.

Here, in the photoimmunotherapy and the photodynamic therapy, since laser light having an output of about 0.5 W to 2.0 W is used, the amount of heat generated in the tubular member 40C through which the laser light L from the optical transmission cable 10 passes is relatively small. Therefore, the heat resistance required for the member is relatively low, and as the material of the tubular member 40C, a material made of a resin more excellent in biocompatibility can be used instead of metal, quartz, or the like. In the photoimmunotherapy and the photodynamic therapy, the optical transmission cable 10 that is a multimode fiber having a relatively large outer diameter d1 of the core 13 of about 500 μm is mainly used. Thus, even if heat sufficient to deform the resin is applied to the tubular member 40C, causing a shift of several μm in the relative position between the emission surface 12 and the refractive surface 31C, optical effects resulting from the shift in the relative position is unlikely to occur. Therefore, the light diffusion device 1 according to the present embodiment uses the tubular member 40C made of resin suitable for use in photoimmunotherapy or photodynamic therapy.

### Fifth Embodiment

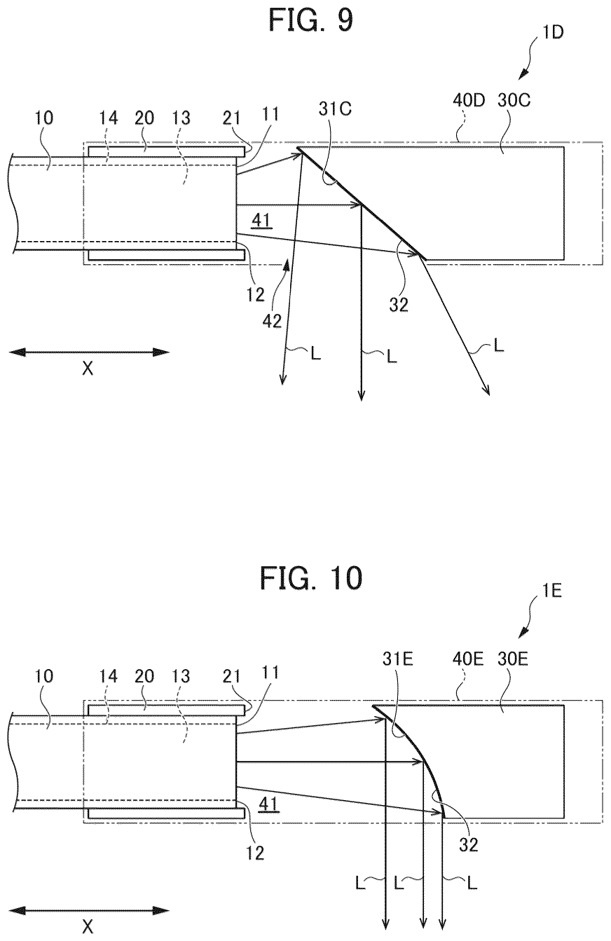

Next, a light diffusion device 1D according to a fifth embodiment will be described with reference to FIG. 9. FIG. 9 is a side view schematically showing the light diffusion device according to the fifth embodiment. In FIG. 9, a tubular member 40D is indicated by a two-dot chain line. In the following description of the fifth embodiment, components corresponding to those of the fourth embodiment are denoted by corresponding reference numerals based on the same conventions. Their descriptions may be omitted or incorporated.

The light diffusion device 1D of the present embodiment includes a laser oscillator (not shown), an optical transmission cable 10, a covering layer 20, a rod-shaped member 30C as a reflective member, and a tubular member 40D. The light diffusion device 1D of the present embodiment mainly differs from that of the first embodiment in the configuration of the tubular member 40D.

The tubular member 40D has an opening 42 formed on the outer periphery thereof. Specifically, the opening 42 is formed in a part of the outer periphery of the tubular member 40D that faces a refractive surface 31C. With this configuration, since the tubular member 40D is not present in the optical path of laser light L emitted from an emission surface 12 via the refractive surface 31C, stronger laser light L can be irradiated to the outside without being transmitted through the tubular member 40D.

### Sixth Embodiment

Next, a light diffusion device 1E according to a sixth embodiment will be described with reference to FIG. 10. FIG. 10 is a side view showing the light diffusion device 1E according to the sixth embodiment. FIG. 10 is a side view of the distal end part side of the light diffusion device 1E in which the structure in the tubular member 40E is also shown. In FIG. 10, the tubular member 40E is indicated by a two-dot chain line. In FIG. 10, some lines are omitted to make the figure easy to see. In the following description of the sixth embodiment, components corresponding to those of the first embodiment are denoted by corresponding reference numerals based on the same conventions. Their descriptions may be omitted or incorporated.

The light diffusion device 1E of the present embodiment includes a laser oscillator (not shown), an optical transmission cable 10, a covering layer 20, a rod-shaped member 30E as a reflective member, and a tubular member 40E. The light diffusion device 1E of the present embodiment mainly differs from the light diffusion device 1C of the fourth embodiment in the configuration of the rod-shaped member 30E.

The rod-shaped member 30E has a refractive surface 31E formed at an end part thereof on the optical transmission cable 10 side. The shape of the refractive surface 31E differs from that of the refractive surface 31C of the rod-shaped member 30C of the fourth embodiment. As shown in FIG. 10, the refractive surface 31E is formed in a curved surface shape that is concave with respect to an emission surface 12 of the optical transmission cable 10. The curvature radius of the refractive surface 31E is preferably 1200 μm. By adjusting the curvature radius of the refractive surface 31E, laser light L emitted from the emission surface 12 can be condensed as well as diffused. For example, as shown in FIG. 10, the laser light L emitted from the emission surface 12 can be uniformly emitted as a whole by the configuration of the curved refractive surface 31E that is concave with respect to the emission surface 12.

### Seventh Embodiment

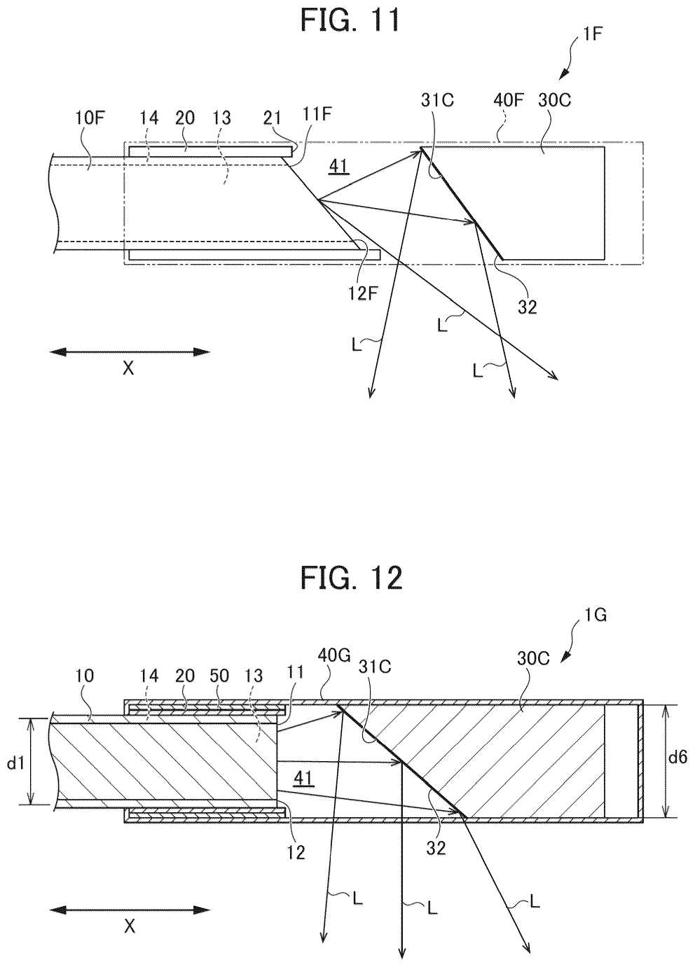

Next, a light diffusion device 1F according to a seventh embodiment will be described with reference to FIG. 11. FIG. 11 is a side view schematically showing the light diffusion device 1F according to the seventh embodiment. FIG. 11 is a side view of the distal end part side of the light diffusion device 1F in which the structure in a tubular member 40F is also shown. In FIG. 11, the tubular member 40F is indicated by a two-dot chain line. In the following description of the seventh embodiment, components corresponding to those of the fourth embodiment are denoted by corresponding reference numerals based on the same conventions. Their descriptions may be omitted or incorporated.

The light diffusion device 1F of the present embodiment includes a laser oscillator (not shown), an optical transmission cable 10F, a covering layer 20, a rod-shaped member 30C, and a tubular member 40F. The light diffusion device 1F according to the present embodiment mainly differs from the light diffusion device 1C according to the fourth embodiment in the configuration of a distal end part 11F of the optical transmission cable 10F.

An emission surface 12F of the optical transmission cable 10F of the present embodiment is formed by cutting the distal end part 11F obliquely with respect to the axial direction X of the optical transmission cable 10F. That is, the emission surface 12F is inclined with respect to the axial direction X of the optical transmission cable 10F. Accordingly, as shown in FIG. 11, laser light L can be emitted from the emission surface 12F while being further diffused. In the present embodiment, as shown in FIG. 11, the emission surface 12F is inclined with respect to the axial direction X of the optical transmission cable 10F so as to face a refractive surface 31C substantially in parallel. Accordingly, the optical transmission cable 10F can be brought closer to the refractive surface 31C, and the laser light L transmitted through the refractive surface 31C without being refracted can be reduced.

### Eighth Embodiment

Next, a light diffusion device 1G according to an eighth embodiment will be described with reference to FIG. 12. FIG. 12 is a side view showing an appearance of the distal end part side of the light diffusion device 1G according to the eighth embodiment. FIG. 12 is a longitudinal sectional view of the distal end part side of the light diffusion device 1G in which the structure in a tubular member 40G is also shown. In the following description of the eighth embodiment, components corresponding to those of the fourth embodiment are denoted by corresponding reference numerals based on the same conventions. Their descriptions may be omitted or incorporated.

The light diffusion device 1G of the present embodiment includes a laser oscillator (not shown), an optical transmission cable 10, a covering layer 20, a rod-shaped member 30C, a tubular member 40G, and an intervening member 50. The light diffusion device 1G of the present embodiment mainly differs from the light diffusion device 1C of the fourth embodiment in that it further includes the intervening member 50 and in the configuration of the tubular member 40G.

The tubular member 40G of the present embodiment is a cylindrical tube made of resin. The tubular member 40G differs from the tubular member 40C of the fourth embodiment in that the inner diameter thereof is slightly smaller than the outer diameter of the rod-shaped member 30C and larger than the outer diameter of the covering layer 20 covering the optical transmission cable 10. The rod-shaped member 30C is housed in the tubular member 40G so that the outer peripheral surface thereof and the inner peripheral surface of the tubular member 40G are in close contact with each other. On the other hand, the covering layer 20 is housed in the tubular member 40G with a space between the outer peripheral surface of the covering layer 20 and the inner peripheral surface of the tubular member 40G.

The intervening member 50 is made of a resin having a low refractive index. The intervening member 50 is disposed along the covering layer 20 in the tubular member 40G, and fills the gap between the outer peripheral surface of the covering layer 20 and the inner peripheral surface of the tubular member 40G. Examples of the resin forming the intervening member 50 include an acrylic resin. The intervening member 50 may be a layer that covers the outer peripheral surface of the covering layer 20, or may be an adhesive that bonds the outer peripheral surface of the covering layer 20 and the inner peripheral surface of the tubular member 40G.

### EXAMPLES

Next, Examples of the present invention will be described. The present invention is not limited to the Examples.

### <Method for Measuring Light Intensity Distribution>

In the Examples, the light intensity distribution of the laser light L emitted from the distal end part of the light transmission cable of the light diffusion device of each of Examples 1 and 2 and a comparative example was confirmed. The light intensity distribution of the laser light L was measured using a beam profiler (SP928, manufactured by Ophir Optronics). The light intensity distribution of the laser light L was obtained by measuring the intensity of the laser light L in a cross section (hereinafter, referred to as a cross section of the laser light L) obtained by cutting the laser light L along a plane orthogonal to the optical axis.

As Example 1, a light diffusion device having the same configuration as that of the light diffusion device 1 of the first embodiment was used. As the optical transmission cable of Example 1, an optical transmission cable having a core with an outer diameter of 500 μm and a cladding with a thickness of 25 μm was used. As the covering layer of Example 1, a nylon tube having a thickness of 50 μm was used. The nylon tube was disposed so as to extend 500 μm from the distal end part of the optical transmission cable 10 in the axial direction X of the optical transmission cable 10.

In Example 2, a light diffusion device having the same configuration as that of Example 1 except for the type of the covering layer was used. In Example 2, a PTFE/polyimide tube was used instead of the nylon tube as the covering layer. The PTFE/polyimide tube used had a PTFE layer thickness of 25 μm and a polyimide tube layer thickness of 25 μm. The PTFE/polyimide tube was disposed so as to extend 500 μm from the distal end part of the optical transmission cable 10 in the axial direction X of the optical transmission cable 10.

As a comparative example, a light diffusion device having the same configuration as that of Example 1 except that a covering layer was not provided was used. Used.

### <Evaluation Results of Light Intensity Distribution>

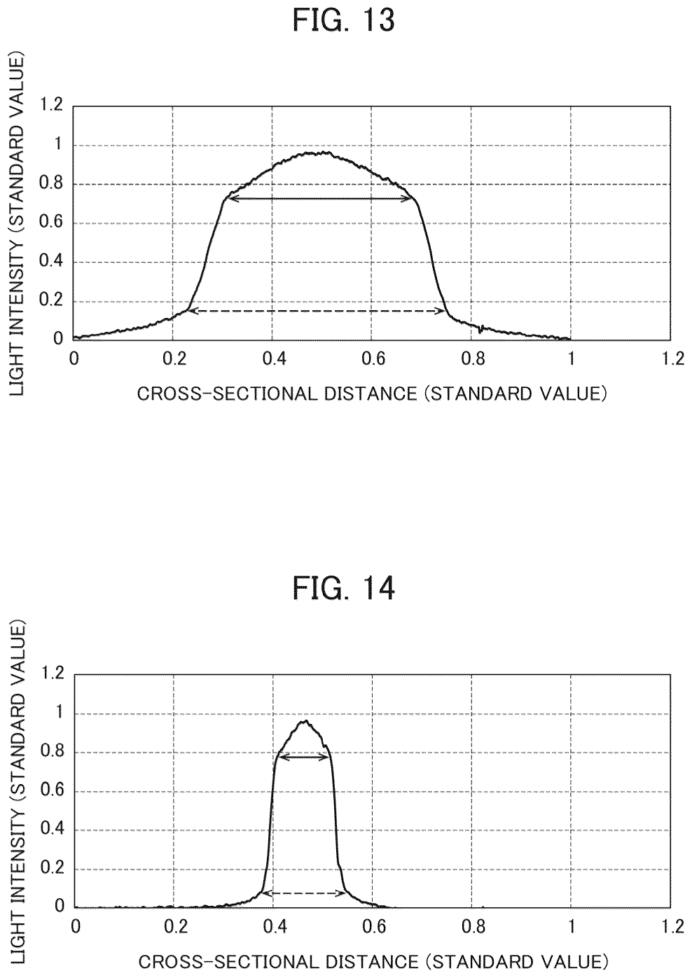

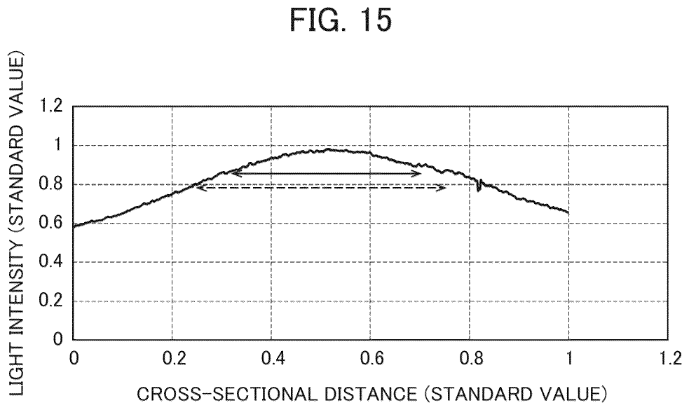

The evaluation results will be described with reference to FIGS. 13 to 15. FIG. 13 is a graph showing a light intensity distribution of laser light emitted from the distal end part of the optical transmission cable of the light diffusion device of Example 1. FIG. 14 is a graph showing a light intensity distribution in the case of using laser light emitted from the distal end part of the optical transmission cable of the light diffusion device of Example 2. FIG. 15 is a graph showing a light intensity distribution in the case of using laser light emitted from the distal end part of the optical transmission cable of the light diffusion device of the comparative example. In FIGS. 13 to 15, the vertical axis indicates the light intensity, and the horizontal axis indicates the cross-sectional distance as the measurement position of the light intensity on the straight line passing through the center of the laser light L in the cross section of the laser light L. The light intensity on the vertical axis in FIGS. 13 to 15 is a standard value in which the maximum value of the measured light intensity is defined as 1, and is a moving average of the measured light intensity, that is, an average value of the light intensity measured within the measurement time. The cross-sectional distance on the horizontal axis in FIGS. 13 to 15 is a standard value in which one end of the measurement position on the straight line is set to 0 and the other end is set to 1. In FIGS. 13 to 15, a range indicated by a solid-line double-sided arrow is a region in which the core exists on the optical transmission cable side in the optical axis direction of the laser light L (hereinafter, referred to as a core region), and a range indicated by a broken-line double-sided arrow is a region in which the core and the cladding exist on the optical transmission cable side in the optical axis direction of the laser light L.

As shown in FIG. 15, in the comparative example, the variation in the light intensity of the laser light L in the core region exceeds 30%. Then, the light intensity gradually decreases and becomes lower as the distance from the center of the laser light L increases. In contrast, as shown in FIGS. 13 and 14, in Examples 1 and 2, it can be confirmed that the variation in of the laser light L in the core region is suppressed to 20% or less on the distal end part side of the optical transmission cable. In the region where the cladding is present on the optical transmission cable side in the optical axis direction of the laser light L, the light intensity of the laser light L sharply decreases as the distance from the center of the laser light L increases. At the position corresponding to the outer periphery of the cladding, it can be confirmed that the light intensity is decreased by 80% or more compared to the light intensity at the center of the laser light L. That is, as shown in FIGS. 13 and 14, it can be confirmed that the light intensity distributions of Examples 1 and 2 provided with a covering layer are more flat-topped than that of the comparative example not provided with a covering layer.

According to the embodiments described above, the following effects are achieved.

The light diffusion devices 1 to 1G for photoimmunotherapy or photodynamic therapy include the optical transmission cables 10, 10A, and 10F configured to transmit the laser light L emitted from a laser oscillator and emit the transmitted light from the emission surfaces 12, 12A, and 12F of the distal end parts 11, 11A, and 11F; and the covering layers 20 and 20A having at least one of a function of absorbing the laser light L or a function of scattering the light, the covering layers 20 and 20A covering the optical transmission cables 10, 10A, and 10F. The covering layers 20 and 20A have distal end parts 21 and 21A that protrude by a required length for cutting the peripheral edge part of the light, in a direction in which the light is emitted. Accordingly, since the laser light L emitted from the peripheral edge side of the distal end parts 11, 11A, and 11F on the distal end parts 11, 11A, and 11F sides of the optical transmission cables 10, 10A, and 10F is removed by the covering layers 20 and 20A and is reflected to the center side of the laser light L, it is possible to emit flat-top laser light L in which the center side of the laser light L is more uniform. Therefore, the light diffusion devices 1A to 1G that irradiate the laser light L with high therapeutic efficiency can be produced by simple processing of forming layers that cover the optical transmission cables 10, 10A, and 10F.

In the light diffusion devices 1 to 1G according to the above embodiments, the optical transmission cables 10, 10A, and 10F each include a core 13 and a cladding 14 formed on the outer periphery of the core 13. The required length Y is calculated by the following Equation (1):

$$Y = {( 1 / {NA}^{2} - 1 )}^{1 / 2} \times d 2 \qquad Equation ( 1 )$$

where Y is the required length, NA is an aperture coefficient of the optical transmission cable, and d2 is the thickness of the cladding. As a result, light that spreads at a wider angle with respect to the optical axis of the laser light L than the spread angle θ of the laser light L can be cut, and light having a flat top light intensity distribution can be more reliably irradiated.

In the light diffusion device 1 according to the above embodiment, the optical transmission cable 10 includes the core 13 and the cladding 14 formed on the outer periphery of the core 13. The cladding 14 has a thickness d2 of 1/10 or less of the outer diameter d1 of the core 13. The covering layer 20 has a thickness d4 greater than that of the cladding 14. Accordingly, it is possible to reduce the diameter of the optical transmission cable 10 while efficiently emitting the laser light L. Even when the cladding mode light leaks to the outside of the cladding 14, since the cladding mode light is removed by the covering layer 20 and is reflected toward the center of the laser light L, more flat-topped laser light L can be emitted.

In the light diffusion devices 1A and 1F according to the above embodiments, the emission surfaces 12A and 12F of the optical transmission cables 10A and 10F are inclined with respect to the axial direction X of the optical transmission cables 10A and 10F. Accordingly, since the laser light L can be irradiated in a direction inclined with respect to the insertion direction of the optical transmission cables 10A and 10F, it is possible to efficiently irradiate the laser light L to cancer cells or the like present on the surface of a long and narrow organ in the human body.

In the light diffusion device 1 according to the above embodiment, the covering layer 20 has a refractive index equal to or greater than the refractive index of the covering material of the optical transmission cable 10. Accordingly, the laser light L emitted from the peripheral edge side of the distal end part 11 can be more reliably absorbed or scattered by the covering layer 20 and reflected toward the center of the laser light L.

In the light diffusion device 1 according to the above embodiment, the covering layer 20 has a refractive index of 1.53 or more. Accordingly, the laser light L emitted from the peripheral edge side of the distal end part 11 can be more reliably absorbed or scattered by the covering layer 20 and reflected toward the center of the laser light L.

The light diffusion devices 1B to 1G according to the above embodiments further include the refractive member 30 having the refractive surface 31, 31C, or 31E that refracts light emitted from the emission surface 12 or 12F, or the rod-shaped member 30C or 30E, and the holding member 40 or one of the tubular members 40C to 40G made of resin into which the optical transmission cable 10 or 10F, and the refractive member 30 or the rod-shaped member 30C or 30E are inserted. The refractive surface 31, 31C, or 31E is disposed at a predetermined distance from the emission surface 12 or 12F in the holding member 40 or one of the tubular members 40C to 40G and is inclined with respect to the axial direction X of the optical transmission cable 10 or 10F. The light emitted from the emission surface 12 or 12F is emitted in a direction that is inclined at a predetermined angle or more with respect to the axial direction X of the optical transmission cable 10 or 10F. Accordingly, flat-top laser light L emitted from the optical transmission cable 10 or 10F can be efficiently irradiated in a direction inclined with respect to the insertion direction of the optical transmission cable 10 or 10F via the refractive surface 31C or 31E. At the time of treatment by photoimmunotherapy or photodynamic therapy, the distal end part 11 or 11F of the optical transmission cable 10 or 10F and the refractive surface 31C or 31E located on the distal end part side of the light diffusion device 1 exposed to the outside from the endoscope are disposed in the holding member 40 or one of the tubular members 40C to 40G made of resin. Accordingly, since it is possible to prevent the relatively hard optical transmission cable 10 or 10F and the refractive surface 31C or 31E made of quartz from coming into contact with organs in the body, biocompatibility is excellent. Further, in addition to the biocompatibility, the degree of freedom of material selection is excellent to meet the needs of device users in terms of cost, operability, etc.

In the light diffusion devices 1B to 1G according to the above embodiments, the refractive member 30 and the rod-shaped members 30C and 30E each are a refractive member 30 or a rod-shaped member 30C or 30E made of quartz or silicon and are spaced apart from the optical transmission cable 10 or 10F in the holding member 40 or one of the tubular members 40C to 40G. The refractive surface 31, 31C, or 31E is formed on an end part of the refractive member 30 or the rod-shaped member 30C or 30E on a side adjacent to the optical transmission cable 10 or 10F. This allows the light diffusion device 1 to be produced more easily.

In the light diffusion devices 1B to 1G according to the above embodiments, the refractive surfaces 31, 31C, and 31E include vapor-deposited metals. This allows light to be refracted more efficiently.

In the light diffusion devices 1B to 1G according to the above embodiments, the optical transmission cables 10 and 10F each are a plastic fiber including the core 13 having an outer diameter of 500 μm or more, and the cladding 14 made of resin and formed on the outer periphery of the core 13. The outer diameters of the refractive surfaces 31, 31C, and 31E viewed from the axial direction X of the optical transmission cables 10 and 10F are larger than the outer diameter of the core 13. Accordingly, since the outer diameters of the refractive surfaces 31, 31C, and 31E are larger than the outer diameter d1 of the core 13, it is possible to improve the tolerance for misalignment of the relative positions of the refractive surfaces 31, 31C, and 31E with respect to the optical transmission cables 10 and 10F.

In the light diffusion devices 1B to 1G according to the above embodiments, the unevenness of the refractive surfaces 31, 31C, and 31E on which the light is incident is equal to or less than the wavelength of the light generated from the light source. Accordingly, since the unevenness of the refractive surfaces 31, 31C, and 31E on which the laser light L is incident is small, it is possible to suppress heat generation by the laser light L on the refractive surfaces 31, 31C, and 31E at the time of irradiation.

In the light diffusion devices 1B to 1G according to the above embodiments, the refractive surfaces 31, 31C, and 31E are formed in a curved surface shape that is concave with respect to the emission surfaces 12 and 12F. Accordingly, since the emission surfaces 12 and 12F of the optical transmission cables 10 and 10F are inclined obliquely, the light emitted from the optical transmission cables 10 and 10F can be further diffused.

Although the embodiments of the present invention have been described above, the present invention is not limited to the above-described embodiments and can be modified as appropriate.

In the above embodiments, the optical transmission cables 10 and 10A include the cladding 14 formed on the outer periphery of the core 13, however, a configuration without the cladding 14 may also be used.

### EXPLANATION OF REFERENCE NUMERALS

1, 1A, 1B, 1C, 1D, 1E, 1F, 1G light diffusion device
10, 10A, 10F optical transmission cable
11, 11A, 11F distal end part
13 core
14 cladding
20, 20A covering layer
21, 21A distal end part

1. A light diffusion device for photoimmunotherapy or photodynamic therapy, the light diffusion device comprising:
an optical transmission cable configured to transmit light emitted from a light source and emit the transmitted light from an emission surface of a distal end part; and
a covering layer having at least one of a function of absorbing the light or a function of scattering the light, the covering layer covering the optical transmission cable,
wherein the covering layer has a distal end part that protrudes by a required length for cutting a peripheral edge part of the light, in a direction in which the light is emitted.

2. The light diffusion device according to claim 1,
wherein the optical transmission cable comprises a core and a cladding formed on an outer periphery of the core, and wherein the required length is calculated by the following Equation (1):
$$Y = {( 1 / {NA}^{2} - 1 )}^{1 / 2} \times d 2 \qquad Equation ( 1 )$$
wherein Y is the required length, NA is an aperture coefficient of the optical transmission cable, and d2 is a thickness of the cladding.

3. The light diffusion device according to claim 1,
wherein the optical transmission cable comprises a core and a cladding formed on an outer periphery of the core,
wherein the cladding has a thickness of 1/10 or less of an outer diameter of the core, and
wherein the covering layer has a thickness greater than that of the cladding.

4. The light diffusion device according to claim 1, wherein the emission surface of the optical transmission cable is inclined with respect to an axial direction of the optical transmission cable.

5. The light diffusion device according to claim 1, wherein the covering layer has a refractive index equal to or greater than a refractive index of a covering material of the optical transmission cable.

6. The light diffusion device according to claim 1, wherein the covering layer has a refractive index of 1.53 or more.

7. The light diffusion device according to claim 1, further comprising:
a reflective member having a refractive surface that refracts the light emitted from the emission surface; and
a tubular member made of resin into which the optical transmission cable and the reflective member are inserted,
wherein the refractive surface is disposed at a predetermined distance from the emission surface in the tubular member and is inclined with respect to an axial direction of the optical transmission cable, and the light emitted from the emission surface is emitted in a direction that is inclined at a predetermined angle or more with respect to the axial direction of the optical transmission cable.

8. The light diffusion device according to claim 7,
wherein the reflective member is a rod-shaped member made of quartz or silicon and is spaced apart from the optical transmission cable in the tubular member, and
wherein the refractive surface is formed on an end part of the rod-shaped member on a side adjacent to the optical transmission cable.

9. The light diffusion device according to claim 7, wherein
the refractive surface comprises a vapor-deposited metal.

10. The light diffusion device according to claim 7,
wherein the optical transmission cable is a plastic fiber comprising a core having an outer diameter of 500 μm or more, and a cladding made of resin and formed on an outer periphery of the core, and
wherein an outer diameter of the refractive surface viewed from the axial direction of the optical transmission cable is larger than the outer diameter of the core.

11. The light diffusion device according to claim 7, wherein an unevenness of the refractive surface on which the light is incident is equal to or less than a wavelength of the light generated from the light source.

12. The light diffusion device according to claim 7, wherein the refractive surface is formed in a curved surface shape that is concave with respect to the emission surface.
